(19) United States
(12) Patent Application Publication
(10) Pub. No.: US 2020/0283734 A1
(43) Pub. Date: Sep. 10, 2020

(54) REPROGRAMMING OF A DIFFERENTIATED CELL TO AN UNDIFFERENTIATED CELL USING EXOSOME

(71) Applicant: AGENCY FOR SCIENCE, TECHNOLOGY AND RESEARCH, Singapore (SG)

(72) Inventors: Karthikeyan NARAYANAN, Singapore (SG); Chan DU, Singapore (SG); Andrew Chwee Aun WAN, Singapore (SG)

(73) Assignee: Agency for Science, Technology and Research, Singapore (SG)

(21) Appl. No.: 16/628,737

(22) PCT Filed: Sep. 7, 2018
(86) PCT No.: PCT/SG2018/050457

§ 371 Date: Jan. 6, 2020

(30) Foreign Application Priority Data
Sep. 8, 2017 (SG) 10201707334T

(51) Int. Cl. C12N 5/074; A61K 35/545

(52) U.S. Cl. CPC C12N 5/0696; C12N 2506/30; A61K 35/545

(57) ABSTRACT

The present invention generally relates to methods of reprogramming cells using exosomes. The present invention also relates methods of screening drugs suitable for cancer treatment using the reprogrammed cells, obtained from the methods described herein. The present invention also relates to therapeutic uses of the reprogrammed cells, obtained from the methods described herein.

### TECHNICAL FIELD

The present invention generally relates to a method of reprogramming a differentiated cell into an undifferentiated cell state using exosome. The present invention also relates to the reprogrammed cell and uses thereof for drug screening and therapy.

### BACKGROUND

The major mediators of cell-cell communications are soluble factors such as cytokines, adhesion molecules, nucleotides and bioactive lipids, which help to coordinate the development and function of tissues and organs. However, additional communication methodologies exist in the form of cellular fragments called exosomes.

Exosomes are lipid membrane vesicles of size ranging from 30-120 nm that are generated by the fusion of endosomal multivesicular bodies and secreted by eukaryotic cells into the extracellular microenvironment. Exosomes contain and transport a variety of biological molecules such as proteins, RNAs and micro RNAs, which can influence the cells that uptake these vesicles. The payloads of the exosomes contribute to various cellular activities such as proliferation and differentiation, and thus, these cellular by-products have immense application in regenerative medicine (1).

The content of the exosomes depends on the physiological state of the secreting cells. As a result, it is highly sought after for biomarker studies to evaluate various pathological conditions such as cancer. Apart from diagnostic use, exosomes have also been used in the area of regenerative medicine. For example, exosomes derived from mesenchymal stem cells were used to initiate pro-angiogenic effects (2). In another study, exosomes from mesenchymal stem cells were shown to have therapeutic effects in various disease models such as myocardial infarction and neural disorders associated with diabetes (3). The common mechanism by which exosomes exert a therapeutic effect in various pathological conditions is via suppression of inflammatory responses and modulation of immune responses (4, 5).

One of the important discoveries of this decade is the reprogramming of somatic cells to a pluripotent state. Reprogramming of fibroblasts with reprogramming factors (Yamanaka factors) has been shown to successfully induce pluripotency in a variety of species (6). Various methodologies have been developed to reprogram fibroblasts, such as single cassette reprogramming vectors with transgenes (7), non-integrating viruses (8), recombinant proteins (9-11), microRNA (miRNA) (12) and messenger RNA (mRNA) (13).

A recent publication suggests the presence of OCT4 protein and mRNA molecules in the extracellular vesicles (including exosomes) isolated from embryonic stem cells (ESCs) conditioned media (14). Moreover, addition of extracellular vesicles to hematopoietic stem cells has a positive effect on their proliferation (15).

It is not known whether exosomes isolated from pluripotent stem cells are capable of reprogramming of somatic cells to a pluripotent phenotype. It is also not known if exosomes isolated from pluripotent stem cells are capable of reprogramming cancer cells to a cancer stem cell (CSC)-like phenotype, which could be useful for studies of drug resistance, and the identification of new candidate drugs that target such cancer stem cell populations. Tumors are made of different types of cancer cells, including CSC. As there is increasing evidence that CSC is more resistant to standard drugs, the use of CSC would lead to more effective anti-cancer therapeutics. Typically, a non-selected cancer cell population (such as those from tumor cells) is used for studies of drug resistance. The non-selected cancer cell population is not a homogenous cell population, being made up of cancer stem cells as well as more differentiated cancer cells, which would not allow screening of a compound that specifically only targets cancer stem cells. In contrast, using a homogenous population of CSC would allow screening of a compound that specifically targets cancer cells, thus leading to a more effective anti-cancer therapeutics.

There is a therefore a need to provide a method of reprogramming a differentiated cell into an undifferentiated cell state (i.e. a reprogrammed cell in an undifferentiated cell state) that overcomes, or at least ameliorates, one or more of the disadvantages described above. The reprogrammed cells could be useful as cell source for regenerative medicine and to establish in vitro models for drug testing.

### SUMMARY

In one aspect, there is provided a method of reprogramming a differentiated cell into an undifferentiated cell state, comprising introducing an exosome from an undifferentiated cell into the differentiated cell. While exosome is known to contain pluripotent transcription factors, it is not known if the concentration of such factors as well as presence of other factors within the exosome would lead to reprogramming, when fed to cells. As a matter of fact, in one example, exosome feeding performed by Zhou et al., 2017 (17) led to a decrease in the expression of pluripotent stem cell markers, decreased proliferation and tumorigenicity of the cancer cells, in contrast to the increased expression of pluripotent stem cell markers (FIGS. 14-15), increased proliferation or clonogenicity (FIG. 13), increased tumorigenicity of the reprogrammed cancer cells (FIG. 20 which shows a faster increase in tumor size from 4 weeks up to 8 weeks for exosome reprogrammed cells (n=4) when compared to control cells (n=3)) and drug resistance (FIGS. 16-17) described in the present disclosure. It was observed that MCF7 and A431 cells show a clear increase in expression of the cancer stem cell markers CD24+ and CD44+ when treated with exosomes.

In another aspect, there is provided an undifferentiated cell population obtained from the method as described herein.

In another aspect, there is provided a method of screening for a candidate agent suitable for cancer treatment, comprising contacting the undifferentiated cell population described herein with a candidate agent and determining the ability of the candidate agent to inhibit the growth of the undifferentiated cell population or kill the undifferentiated cell population. Advantageously, the reprogrammed cancer cells of the present disclosure may have acquired the stem cell-associated characteristic of drug resistance. For example, FIGS. 16-17 show that EX-iCSCs may be more resistant to sunitinib and doxorubicin compared to non-reprogrammed cells. The screening for a compound that selectively kills the EX-iCSCs could also lead to the identification of a compound that is more effective in killing the cancer stem cell/tumor-initiating cancer cell population in vivo, leading to identification of a more effective cancer drug.

In another aspect, there is provided an undifferentiated cell population described herein for use in therapy

### BRIEF DESCRIPTION OF THE DRAWINGS

The invention will be better understood with reference to the detailed description when considered in conjunction with the non-limiting examples and the accompanying drawings, in which:

FIG. 1A is a schematic diagram showing exosome isolation from pluripotent stem cells and its use for fibroblast reprogramming to obtain iPSC-like cells (exosome-reprogrammed cells). FIG. 1B is a scanning electron microscope (SEM) image of exosomes isolated from pluripotent stem cell line, HUES-7 (Bar=1 μm). FIG. 1C is a transmission electron microscope (TEM) image of exosomes isolated from pluripotent stem cell line, HUES-7 (Bar=10 nm). FIG. 1D is a table summarizing the results of particle size analysis for exosomes from different sources. Typically, the average counted particles per frame is between 50 and 300, while the number of traced particles is between 1000 and 7000.

FIG. 2A shows the relative expression of mRNAs of the NANOG, FGF2 and OCT3/4 genes (determined by RT-PCR; n=3) in exosomes isolated from different pluripotent stem cell sources. FIG. 2B is a table showing the expression level of specified genes in the exosomes compared to that for total RNA isolated from iPSC (IMR-90), in terms of Ct values. FIG. 2C shows a dot blot analysis of protein molecules associated with reprogramming factors (Lane 1: iPS cells, Lane 2: exosomes from iPS cells, Lane 3: exosomes from H1 cells, Lane 4: exosomes from H7 cells). FIG. 2D shows the miRNA profile of the HUES7 exosomes (n=3).

FIG. 3A shows fluorescence microscopy images showing the uptake of labelled exosomes (from HUES7) by human fibroblasts (IMR-90) (Bars=200 μm). FIG. 3B shows fluorescence microscopy images showing the uptake of labelled exosomes by human fibroblasts at higher magnification (Bars=200 μm). FIG. 3C shows the fluorescence intensity measurement of exosome uptake (n=3).

FIG. 4 shows the exosome reprogramming of IMR-90-OCT3-GFP fibroblasts. FIG. 4A shows the POU5f1 (OCT3) promoter-driven GFP vector for monitoring cell reprogramming process. FIG. 4B shows the images of colonies that expressed OCT3 and which were transferred to MEF layer, and subsequently, Matrigel, for further culture (under brightfield, under fluorescent microscopy to detect the GFP signals and a merged composite of the brightfield and fluorescent images) (Bars from top to bottom: First row=100 μm; Second row=50 μm; Third row: 100 μrn; Fourth row: 500 μm).

FIG. 5 shows the gene expression analysis of key pluripotent genes at different passages of exosome reprogramming (n=3).

FIGS. 6A, 6B and 6C show the flow cytometry for pluripotent marker expression in HUES7, EX-iPSC-1 and EX-iPSC-2, respectively.

FIG. 7 shows immunostaining of exosome reprogrammed cells (iPSC-ex-1) for pluripotent stem cell markers, with nuclear co-staining using DAPI (Bars=100 μm).

FIG. 8 shows immunostaining of iPSC-ex-2 cells, showing the expression of pluripotency markers (Bars=100 μm).

FIG. 9 shows microscopy images of in vivo teratoma formation by exosome-reprogrammed cells (EX-iPSC-2), showing multiple cell types such as chondrocytes in FIG. 9A, lung epithelial cells with blood vessels in FIG. 9B, intestinal epithelial cells with blood vessels in FIG. 9C, and glandular columnar cells in FIG. 9D. Arrows indicate the blood vessels (Bars: A and B=100 μm; C and D=200 μm).

FIG. 10 shows microscopy images of in vivo teratoma formation by exosome-reprogrammed cells (EX-iPSC-1), showing multiple cell types (Bars=200 μm). Arrows indicate the blood vessels.

FIG. 11 shows directed differentiation of EX-iPSCs to neural lineage. FIG. 11A shows immunostaining of differentiated cells with neural stem cell markers (Bars=100 μm). FIG. 11B shows gene expression of neural markers for cells obtained by differentiation of EX-iPSC-1, EX-iPSC-2 and iPS cells, respectively (n=3).

FIG. 12 shows karyotype analysis for fibroblasts and exosome-reprogrammed cells.

FIG. 13A shows the colony forming assay for exosome-treated MCF7 cancer cells (Bars=200 μm). FIG. 13B shows the number of colonies formed by MCF7 cells when treated with exosomes from different sources, compared with the non-treated control. FIG. 13C shows the number of colonies formed by A431 cells when treated with exosomes from different sources, compared with non-treated control (n=3).

FIG. 14A shows the flow cytometry analysis of CD24+ and CD44+ populations for exosome-treated cancer cell line MCF7. FIG. 14B shows the flow cytometry analysis of CD24+ and CD44+ populations for exosome-treated cancer cell line A431. FIG. 14C shows the flow cytometry analysis of CD24+ and CD44+ populations for exosome-treated cancer cell line DLD-1. FIG. 14D shows the flow cytometry analysis of CD24+ and CD44+ populations for exosome-treated cancer cell line and (D) MDA-MB-231.

FIG. 15 shows tables summarizing flow cytometry analysis of CD24+ and CD44+ populations for exosome-treated cancer cell lines.

FIG. 16 shows cell viability of exosome-treated A431 cancer cell line (A431 EX-iCSCs) when treated with the cancer drugs, cisplatin, doxorubicin and sunitinib, respectively. Horizontal axes represent the drug concentrations in μg/mL.

FIG. 17 shows cell viability of exosome-treated MCF7 cancer cell line (MCF7 EX-iCSCs) when treated with the cancer drugs, cisplatin, doxorubicin and sunitinib, respectively. Horizontal axes represent the drug concentrations in μg/mL.

FIG. 18 lists the Taqman assays used in the study.

FIG. 19 lists the antibodies used in the study.

FIG. 20 shows the increased tumorigenicity of the reprogrammed cancer cells (shaded bars) in vivo compared to control cells (non-shaded bars).

### DEFINITION OF TERMS

The term “reprogramming” when used in relation to cellular reprogramming as described in the present disclosure refers to the process of converting a differentiated cell (such as a somatic cell or a cancer cell) into a cell having an undifferentiated state. In one example, a cell having an undifferentiated state may be referred to as an undifferentiated cell. The undifferentiated cell resulting from the reprogramming may also be a pluripotent cell or a cancer stem cell-like cell. In another example, “reprogramming” refers to the process of converting a differentiated cell, such as a somatic cell, into a pluripotent cell by introducing exosomes from an undifferentiated cell into the somatic cell. In another example, “reprogramming” refers to the process of converting a differentiated cell, such as a cancer cell, into a cancer stem cell-like cell by introducing exosomes from an undifferentiated cell into the cancer cell.

A “reprogramming factor” refers to a protein expressed by a specific pluripotency-associated gene.

By “introducing” an exosome into a cell, it is generally meant as delivery of the exosome by various means available in the art, such as by clathrin-mediated endocytosis, phagocytosis, membrane fusion, caveolin-mediated endocytosis, micropinocytosis, lipid raft-mediated endocytosis, and combinations thereof, into the cell.

The term “contact”, or grammatical variations thereof, as used in the context of the methods of the present disclosure, relates to bringing a substance, such as a cell, of the present disclosure in physical contact with another substance, such as a cell organelle like exosome, or otherwise exposing the cell to the other substance, such as cell organelle like exosome, to thereby allow uptake of the substance into the cell. The term may also relate to bringing a substance, such as a cell, of the present disclosure in physical contact with another substance, such as a candidate drug agent, or otherwise exposing the cell to the candidate drug agent, to thereby allow uptake of the candidate drug agent into the cell.

The term “treatment” refers to any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment.

The term “differentiated cancer cell” refers to cancer cells which look like normal cells from the tissue which they are isolated from. Differentiated cancer cells tend to proliferate and spread more slowly than poorly differentiated or undifferentiated cancer cells. Differentiated cancer cells have the ability to acquire mutations or activate a transcription factor, or otherwise be reprogrammed into an undifferentiated state, such as a CSC or a CSC-like state.

The term “somatic cell” refers to any cell of a living organism which is not a reproductive cell or germ cell.

The term “pluripotent cell” refers to cells that can give rise to all the cell types that make up the body. Exemplary pluripotent stem cells include embryonic stem cells, and iPSC which are genetically reprogrammed to assume a stem cell-like state.

The term “cancer stem-cell like cell” refers to a cancer cell which possesses cancer stem cell characteristics such as the ability to self-renew and ability to give rise to differentiated cancer cells.

The term “inhibit” when used in relation to the method of screening for candidate agents for cancer therapy refers to hindering or preventing growth of a cell, such as a cancer cell or a cancer stem-cell like cell. The inhibition may be total inhibition or partial inhibition, such that about 10%, about 20%, about 30%, about 40%, about 50%, about 60%, about 70%, about 80%, about 90% or 100%, of growth is inhibited or prevented.

The word “substantially” does not exclude “completely” e.g. a composition which is “substantially free” from Y may be completely free from Y. Where necessary, the word “substantially” may be omitted from the definition of the disclosure.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DESCRIPTION OF THE PRESENT INVENTION

Pluripotency, the ability to become any cell type, is a valuable cellular characteristic. Induced pluripotent cells are generated by reprogramming a differentiated somatic cell back into its undifferentiated embryonic state. These cells are able to form teratomas (which differentiate into all three germ layers). Induced pluripotent cells may be useful for replacing damaged tissues or for studying diseased cells towards the development of a treatment for the diseases. The advantage of using induced pluripotent cell generated from somatic cells of a patient is that since the somatic cell is from the patient, the genetic makeup of the induced pluripotent cell would theoretically be similar to the patient's cell. Methods known in the art such as single cassette reprogramming vectors with transgenes (7), non-integrating viruses (8), recombinant proteins (9-11), microRNA (miRNA) (12) and messenger RNA (mRNA) are technically more complicated and cumbersome ways to generate induced pluripotent stem cells from somatic cells. Methods involving cumbersome transgene integration methods may also lead to genomic instability in the cells. Thus, the present disclosure provides a technically less complicated alternative as the present method requires only the presence of exosomes in the culture media.

OCT4 protein and mRNA (a key regulator in pluripotency) were previously discovered in the exosomes of embryonic stem cells. Exosomes can also influence the cells that successfully uptake the exosomes vesicles.

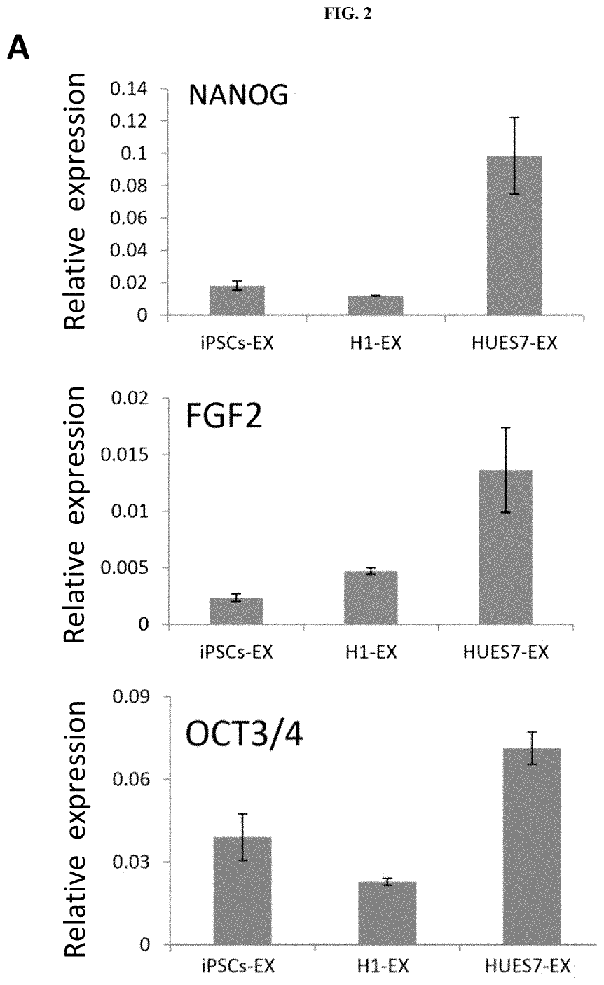

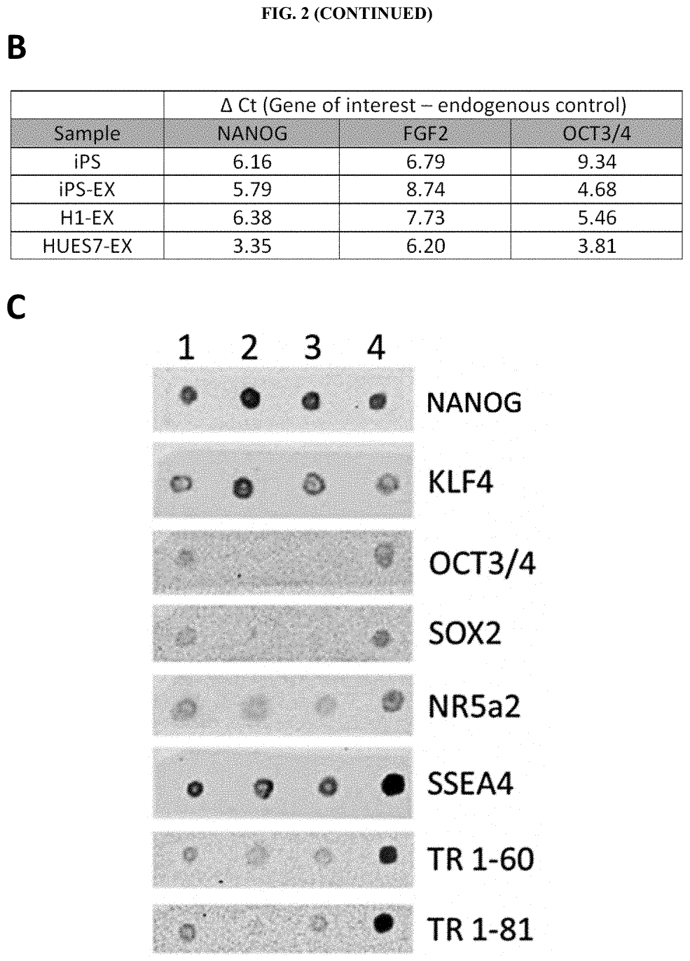

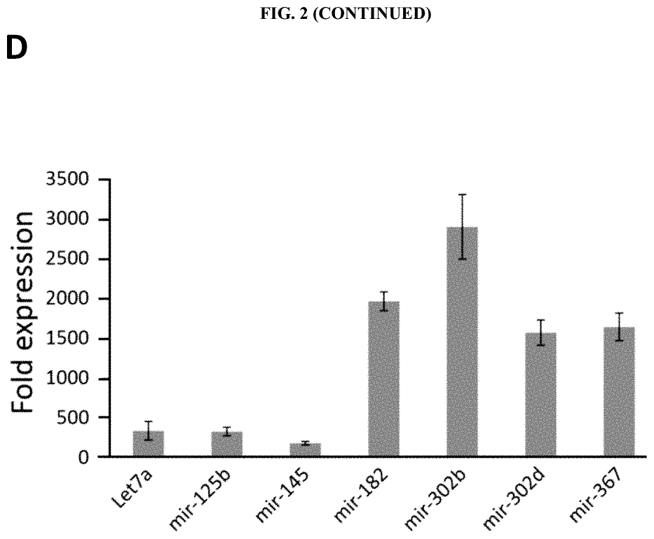

In one example, the inventors have identified the exosomes of HUES-7 to have high expression of reprogramming factors (NANOG, FGF2, and OCT2) compared to the exosomes from IMR-90 or the H1 human embryonic stem cell line (FIG. 2). Therefore, the exosomes of HUES-7 can be useful in reprogramming a differentiated cell (such as a somatic cell) into an undifferentiated cell, such as a pluripotent cell.

Thus, in one aspect, there is provided a method of reprogramming a differentiated cell into an undifferentiated cell state, comprising introducing an exosome from an undifferentiated cell into the differentiated cell.

The exosome may be introduced into the differentiated cell by contacting the differentiated cell with the exosome from the undifferentiated cell in a first medium suitable for uptake of the exosome by the differentiated cell, thereby reprogramming the differentiated cell into the undifferentiated cell state.

The composition of the first medium is designed to allow for efficient uptake of the exosomes by the differentiated cell. The first medium in which exosomes is contacted with the differentiated cell can be, but is not limited to, a composition comprising Dulbecco's Modified Eagle Medium/Nutrient Mixture F-12 (DMEM/F12), Penicillin-Streptomycin (P/S) and 10% Fetal Bovine Serum (FBS).

The time required for contacting the exosome with the differentiated cell to enable sufficient uptake of exosome by the differentiated cell would depend on various factors such as the source of the exosome, the differentiated cell type into which the exosome is introduced, the composition of the medium used, etc., and may be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, or at least 16 days. In one example, the contacting can occur for 3 days.

The method described herein may also further comprise incubating the reprogrammed cells in a second medium. The composition of the second medium is designed to provide suitable nutrients for the reprogrammed pluripotent cells to propagate. In one example, the second medium in which the reprogrammed pluripotent cell is incubated in is ESGRO-2i. Other suitable second medium may be such as, but not limited to, DMEM. It was found that ESGRO-2i is suitable as the second media for the culture of the reprogrammed somatic cells. On the other hand, DMEM is suitable as the second media for the reprogrammed cancer cells.

The incubation time of the reprogrammed cell in the second medium to enable the reprogrammed cells to propagate to the desired level may be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, or at least 16 days. In one example, the incubation time in the second medium is 7 to 10 days. The incubation time of the reprogrammed cell in the second medium to enable the reprogrammed cells to propagate to the desired level may be at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

In one example, the exosomes used for the method as described herein for reprogramming a differentiated cell are isolated using any methods known in the art for isolation of cell organelles, for example from a stem cell from human or non-human mammals. The exosomes may be isolated through centrifugation. Suitable non-human mammals that fall within the scope of the disclosure include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

In one example, the stem cell is selected from a group consisting of embryonic stem cell (ESC), induced pluripotent stem cell (iPSC), and embryonic germ cell.

Exosomes may be isolated from any embryonic stem cell or induced pluripotent stem cells. The embryonic stem cells may be obtained from cell lines such as the H1, H7, HUES-7, and H9 cell lines. The induced pluripotent stem cells may be IMR-90-iPSC.

The exosome may contain biomolecules such as reprogramming factors, and miRNAs, which have reprogramming ability. In one example, the exosome contains one or more pluripotent factors selected from a group consisting of NANOG, FGF2, OCT3/4, SOX2, NR5a2, SSEA-4, TR-1-60, and TR-1-81. In another example, the exosome contains one or more miRNAs selected from the group consisting of Let7a, mir-125b, mir-145, mir-182, mir-302b, mir-302d, and mir-367.

The inventors had found that exosomes isolated from HUES7 showed higher expression levels of the reprogramming proteins (such as reprogramming factors NANOG, FGF2, OCT3/4, and SOX2), RNAs and miRNAs (see FIG. 2B, 2C, 2D), and may therefore be suitable for use in reprogramming of differentiated cells.

The differentiated cell may be a somatic cell. The somatic cell can be any somatic cell which may be obtained using standard methods known in the art, from human or other mammals. For example, the somatic cell can be, but is not limited to, fibroblast, somatic stem cell, sertoli cell, endothelial cell, neuron, pancreatic islet cell, epithelial cell, hepatocyte, hair follicle cell, keratinocyte, hematopoietic cell, melanocyte, chondrocyte, lymphocyte, erythrocyte, macrophage, monocyte, mononuclear cell, muscle cell, and combinations thereof.

Suitable fibroblast for reprogramming can be, but is not limited to, lung fibroblast, dermal fibroblast, bladder fibroblast, uterine fibroblast, vas deferens fibroblast, and combinations thereof. In one example, the lung fibroblast is human lung fibroblast IMR-90. In one example, the epithelial cell is a granulosa epithelial. In one example, the muscle cell is a cardiac muscle cell.

In another example, the inventors have also shown that exosomes from the HUES-7 embryonic stem cell line were able to reprogram not only somatic cells, but also cancer cells.

The cancer cell can be, but is not limited to breast cancer cell, colorectal cancer cell, epidermoid cancer cell, epithelial tissue cancer cell (carcinoma cell), connective tissue cancer cell (sarcoma cell), blood cancer cell, cancer cell from the lymphatic system (lymphoma cell), lung cancer cell, skin cancer cell, and combinations thereof. The breast cancer cell can be, but is not limited to MCF7, MDA-MB-231, and BT474 cell. The colorectal cancer cell can be, but is not limited to, colon carcinoma cell, rectal cancer cell and colorectal sarcoma cell. The colon carcinoma cell can be, but is not limited to DLD-1, SW1116, Caco-2, SW480. The epidermoid cancer cell can be, but is not limited to A431, and AW8507 cell. The carcinoma cell can be, but is not limited to, adenocarcinoma cell, basal cell carcinoma cell, squamous cell carcinoma cell and transitional cell carcinoma cell. The sarcoma cell can be, but is not limited to, soft tissue sarcoma cell, chondrosarcoma cell, rhabdomyosarcoma cell and leiomyosarcoma cell. The blood cancer cell can be, but is not limited to, acute lymphocytic leukemia cell, acute myelogenous leukemia cell, chronic lymphocytic leukemia cell and chronic myelogenous leukemia cells. The lymphoma cell can be, but is not limited to, Hodgkin lymphoma cell and non-Hodgkin lymphoma cell. The skin cancer cell can be, but is not limited to, superficial spreading melanoma cell, lentigo maligna cell, acral lentiginous melanoma cell and nodular melanoma cell.

The undifferentiated cell state may be a pluripotent cell state or a cancer stem cell-like cell state.

In one example, the undifferentiated cell is in a pluripotent cell state. Such a cell has one or more of the following characteristics:

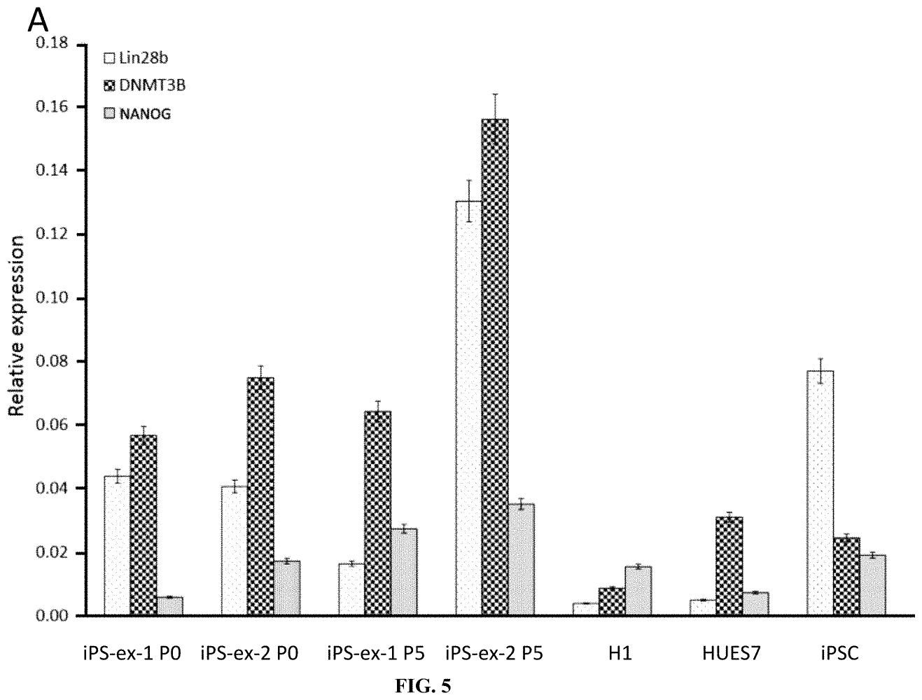

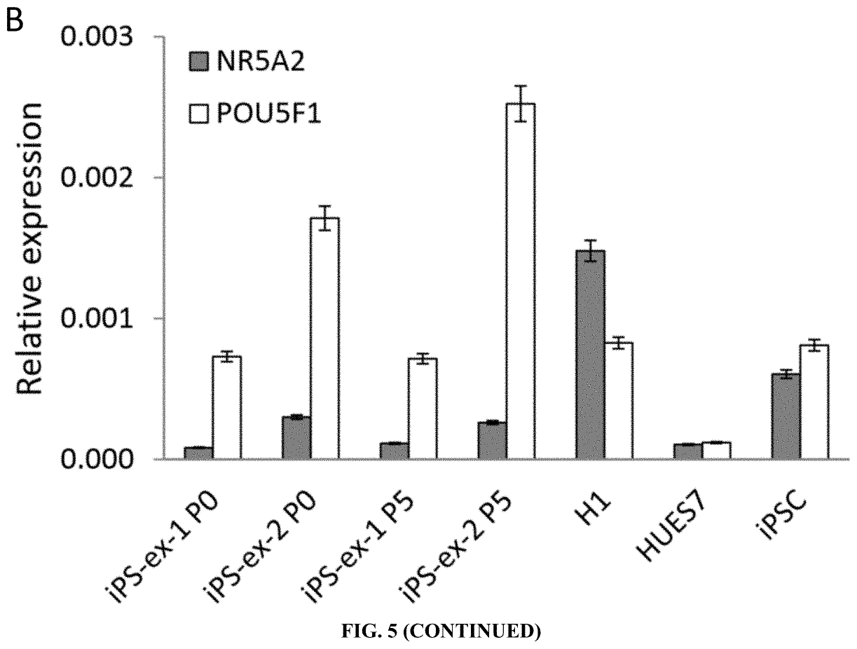

(i) has similar gene and surface marker expression profile as that of an embryonic stem cell and an induced pluripotent stem cell, such as shown in FIG. 5;

(ii) is pluripotent; and

(iii) is able to form three germ layer tissues.

The term “surface marker” refers to proteins that are expressed on the surface of a cell which can serve as markers of specific cell types. The presence of these markers on the surface allows convenient identification of the specific cell type using methods common in the art which may be, but are not limited to, image-based flow cytometry. Other non-image-based quantitative means of detection of protein expression may be, but are not limited to, real-time PCR, microarray, RNA Seq, Serial Analysis of Gene Expression (SAGE), Western blotting and Northern blotting. The term “similar gene and surface marker expression profile” refers to expression of similar sets of genes, such as upregulation and/or downregulation of the same sets of genes between two or more samples. A gene may be considered upregulated if its expression is increased by more than 5%, more than 10%, more than 20%, more than 30%, more than 40%, more than 50%, more than 60%, more than 70%, more than 80%, more than 90%, more than 100%, more than 110%, more than 120%, more than 130%, more than 150%, more than 200%, or more than 300% compared to the expression level of the gene in a control sample. A gene may be considered downregulated if its expression is decreased by more than 5%, more than 10%, more than 20%, more than 30%, more than 40%, more than 50%, more than 60%, more than 70%, more than 80%, more than 90%, or 100% compared to the expression level of the gene in a control sample. Alternatively, a gene may be considered to be upregulated if the expression level is significantly higher than the gene and surface marker expression level of the control sample when determined using statistical tests. A gene may be considered to be downregulated if the expression level is significantly lower than the gene and surface marker expression level of the control sample when determined using statistical tests. Some examples of statistical tests are t-statistic, the efficiency calibrated model, and the ΔΔCt model.

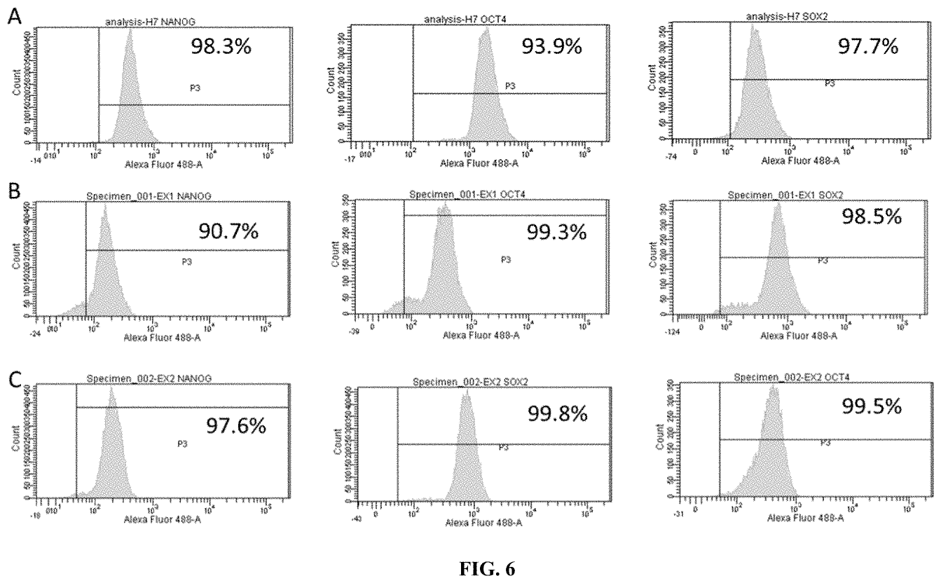

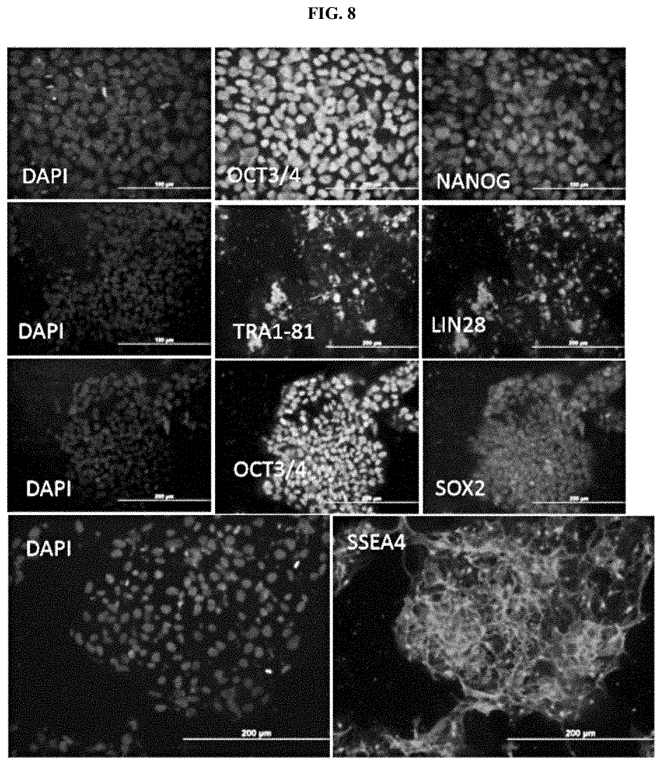

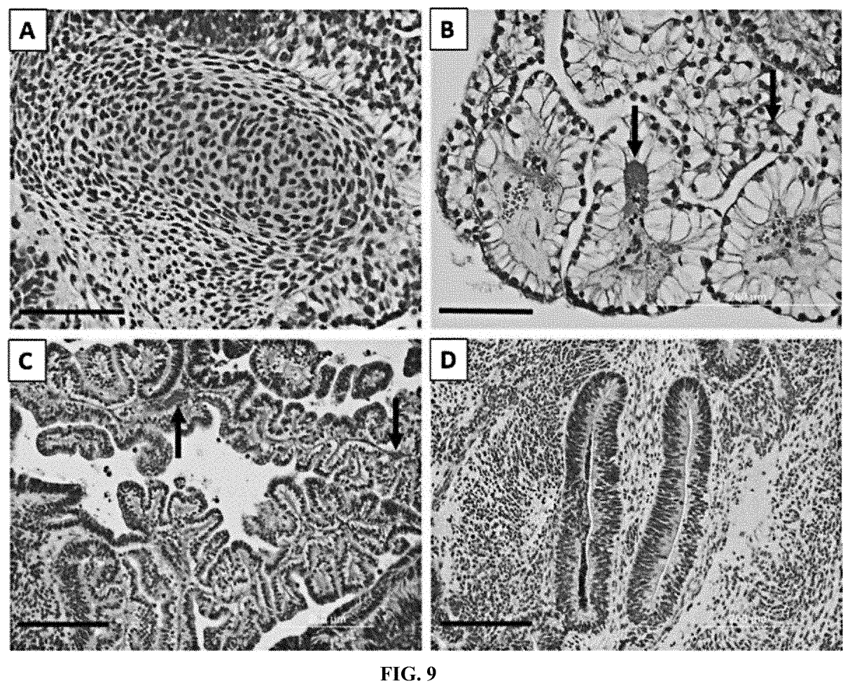

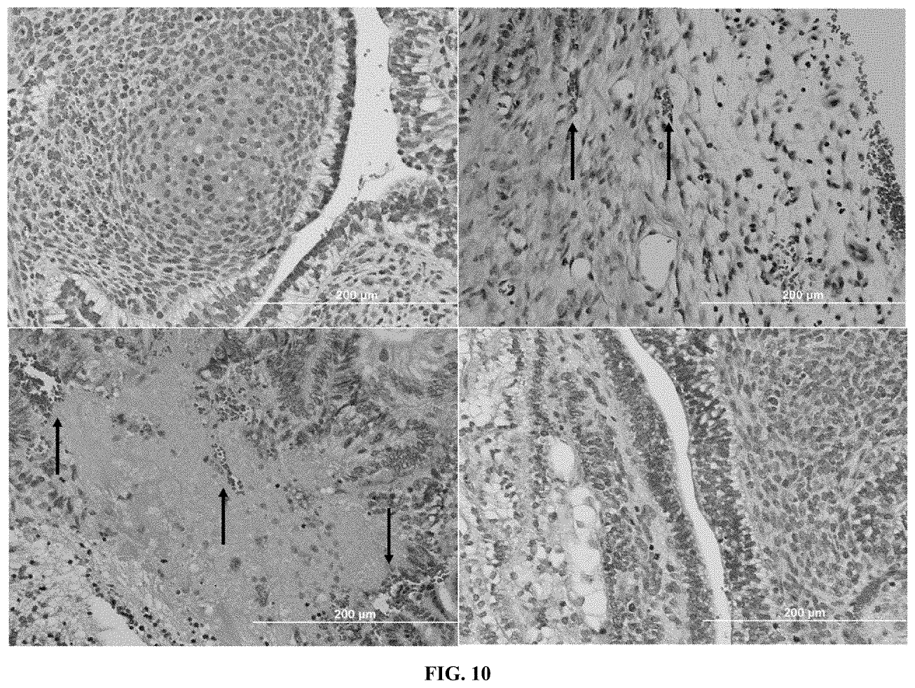

The resulting undifferentiated cell populations, obtained from a differentiated cell (such as somatic cells) by the method described herein include those in the examples (EX-iPSC-1 and EX-iPSC-2). Both populations show high levels of gene expression for the pluripotent factors NANOG, OCT4 and SOX2 (FIG. 6). Immunostaining with antibodies of OCT3/4, Nanog, TRA1-81, Lin28, SOX2 and SSEA-4 showed correct localization of the respective proteins in the exosomes-reprogrammed EX-iPSC-1 and EX-iPSC-2 cells (FIG. 8). The pluripotent cells obtained from the method as described herein also show embryonic stem-cell like ability to differentiate into the three primary germ layers (FIG. 9 and FIG. 10).

Other genes of the undifferentiated cells in the pluripotent state obtained from the method as described herein which may be expressed with similar profiles as that of an embryonic stem cell and an induced pluripotent stem cell may be, but not limited to, OCT3/4, NANOG, TRA-1-81, Lin28, SOX2, SSEA-4, SSEA-1, TRA-1-60, Sall4, Dax1, Essrb, Tbx3, Tcl1, Rif1, Nac1 and Zfp281.

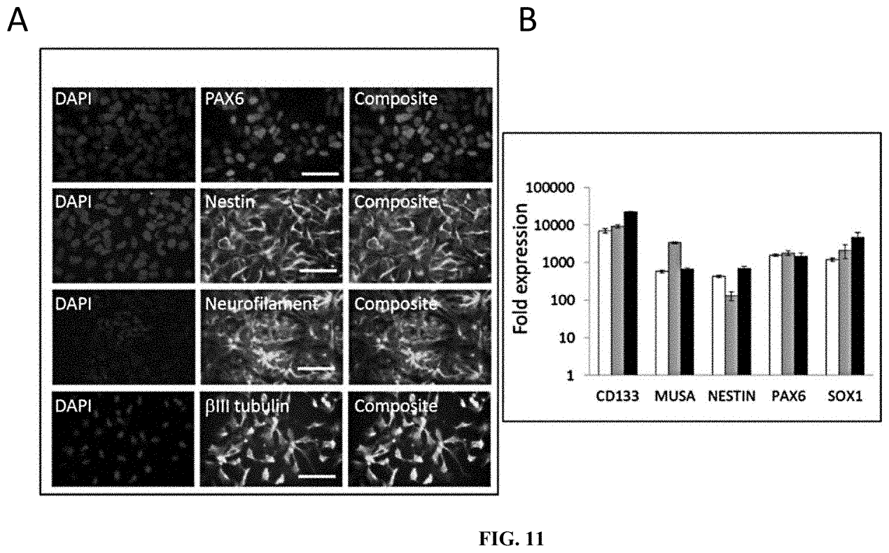

The exosomes-reprogrammed cells obtained from the method as described herein were demonstrated to be able to differentiate into cells of a different lineage and therefore could be used for therapy. In one example, the reprogrammed cells undergo directed differentiation towards the neural lineage, as shown by up-regulation of neural stem markers (such as CD133, MUSASHI, NESTIN, PAX6 and SOX1) and expression of neural lineage markers (such as PAX6, nestin, neurofilament and β3 tubulin) (FIG. 11). The differentiated cells of the neural lineage could find further use in cell-based therapy of spinal cord defects, while dopamine-producing neural cells could be used for Parkinson's disease therapy.

In one example, the undifferentiated cell is in a cancer stem cell-like cell state. Such a cell has one or more of the following characteristics:

(i) has similar gene and surface marker expression profile as a cancer stem cell; and

(ii) has similar drug resistance characteristics as a cancer stem cell.

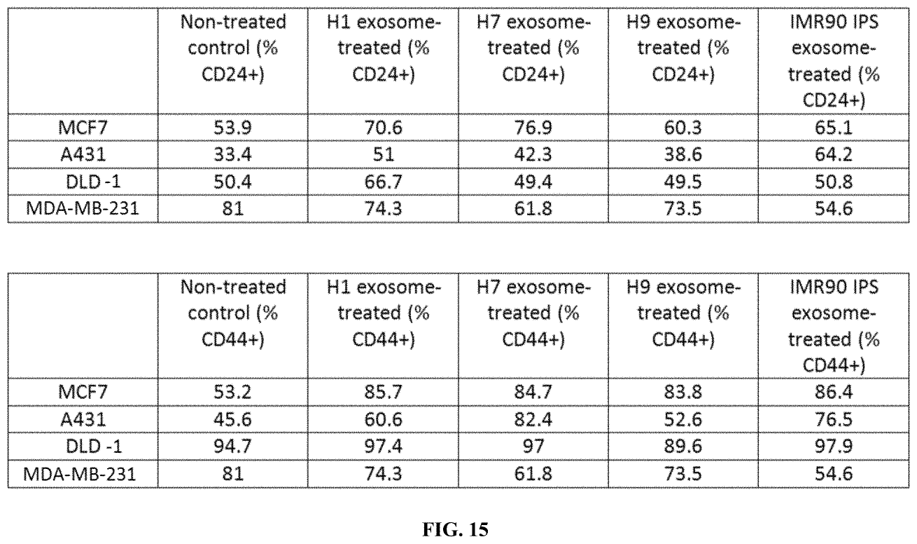

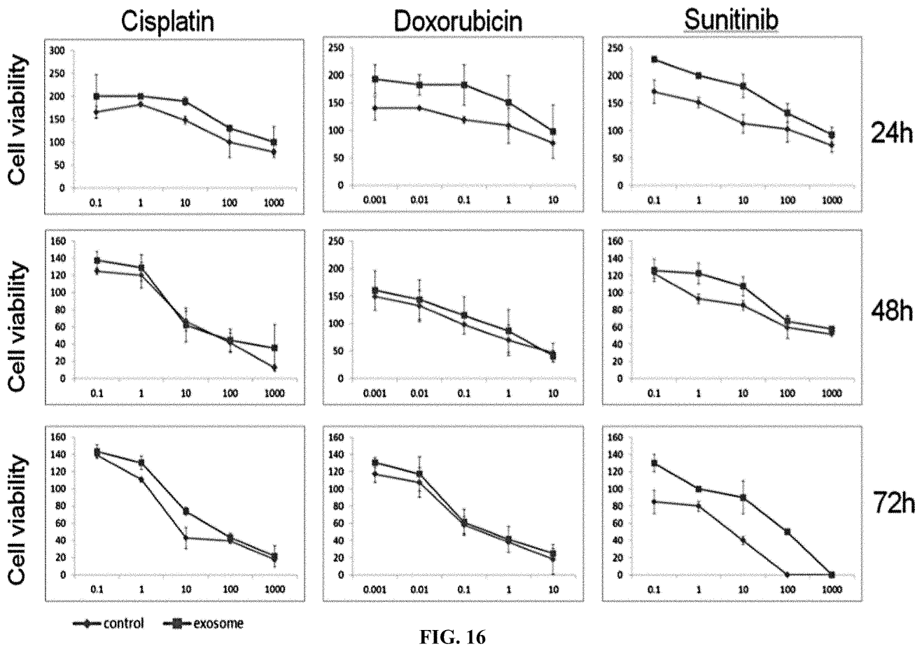

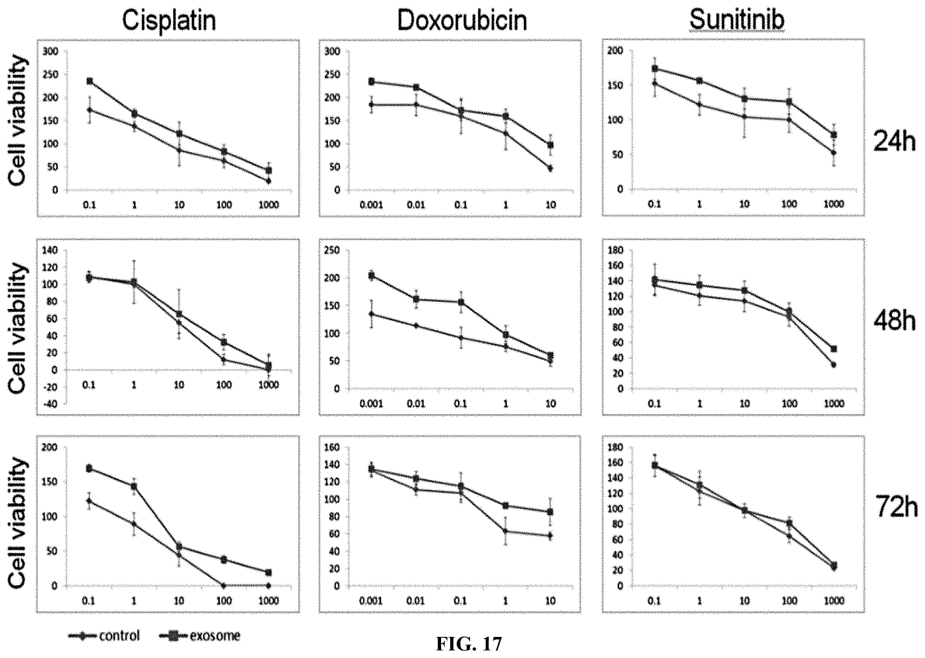

The resulting undifferentiated cell populations, obtained from differentiated cells (such as cancer cell) by the method described herein include those in the examples (designated as EX-iCSCs). These reprogrammed cancer cells were shown to have one or more cancer stem cell (CSC) markers, such as CD24 and CD44. These two markers, which are cancer stem cell-related surface markers were found to be expressed in the EX-iCSCs obtained by the method described herein (FIG. 15). The cells may also show other characteristics of CSC, such as drug resistance. In one example, the EX-iCSCs showed drug resistance characteristics towards cancer drugs (such as cisplatin, doxorubicin, sunitinib), a characteristic which has been associated with stem cell-like traits in cancer cells (FIG. 16, FIG. 17).

Other genes of the undifferentiated cells in cancer stem cell-like cell state obtained from the method as described herein which may be expressed with similar profiles as that of a cancer stem cell may be, but not limited to, CD24, CD44, CD133, CXCR4, c-Met, ALDH1, and ABCG2.

In another aspect, there is provided an undifferentiated cell population obtained using the method as described herein. The undifferentiated cell population may exhibit some or all of the cell characteristics as described above.

The undifferentiated cell population may be used in various applications.

Thus, in one aspect, there is provided a method of screening for a candidate agent suitable for cancer treatment, comprising contacting the undifferentiated cell population obtained using the method as described herein with a candidate agent and determining the ability of the candidate agent to inhibit the growth of the undifferentiated cell population or kill the undifferentiated cell population. If the candidate agent is able to inhibit the growth of the undifferentiated cell population or kill the undifferentiated cell population, then the candidate agent may be a promising drug for cancer therapy.

In another aspect, the undifferentiated cell population obtained using the method described herein may be used in therapy. Therapeutic uses could include further differentiating the undifferentiated cell population (obtained using the method described herein) into the desired specific differentiated cell population. The differentiated cells can be transplanted to address a functional deficit in the damaged or diseased tissues of the same type or characteristics. For example, an undifferentiated cell prepared using the method described above may undergo directed differentiation into a differentiated cell of a specific lineage, such as neural lineage, which can then be used for treating conditions such as spinal cord defect or Parkinson's disease.

### EXAMPLES

### Example 1: Material and Methods

### Cell Culture

Human lung fibroblasts (IMR-90, product code CCL-186) were obtained from (American Type Culture Collection (ATCC), VA, USA). Cells were cultured at 37° C. with 5% C0₂ in Dulbecco's modified Eagle's medium (DMEM) containing 4.5 mg/ml glucose, supplemented with 10% heat inactivated fetal bovine serum, 2 mM L-glutamine, 50 μg/ml penicillin and 50 μg/ml streptomycin. Cultures at ˜80% confluence were routinely split using trypsin.

Primary normal neonatal human dermal fibroblasts of foreskin origin were purchased from American Type Culture Collection (ATCC), VA, USA (product code ATCC® PCS-201-010™). The cells were maintained with Fibroblast Growth Kit-Low Serum (ATCC), and cells of passage number less than five were used for the experiments listed in the present disclosure.

### Pluripotent Stem Cell Culture

Human embryonic stem cells H1, H7 and HUES-7 and human induced pluripotent stem cells (IMR-90 derived) (9) were used in the study. HUES-7 cell line was obtained from Harvard University (MA, USA). Other cell lines H1, H7, H9 and IMR-90-iPSC were purchased from WiCell Research Institute (WI, USA) (product codes: WA01, WA07, WA09 and iPS(IMR90) respectively). Pluripotent stem cells were cultured on a feeder-free system. Matrigel (BD Biosciences, USA) coated plates were used for the culture of the pluripotent stem cells with mTesR1 media. Media was changed every 24 hr. Unwanted differentiated cells were physically removed by scraping with Pasteur pipette. Dispase was used to subculture the cells. Conditioned media was collected from cells grown to a confluence of 70% every day for 5 days, and processed for exosome isolation.

Isolation of Exosomes from ES Cell Conditioned Medium

The pluripotent stem cells were cultured on 150 mm (diameter) petri dishes coated with Matrigel as mentioned earlier. Upon achieving 70% confluence, conditioned media was collected for exosome isolation. The collected media was cleared of cellular debris by a brief centrifugation at 2800 g for 20 min. The obtained supernatant was subjected to further centrifugation at 100000 g for 70 mins. The exosome pellet was washed with PBS and collected by centrifugation at 100000 g for 70 mins. All the centrifugations were performed at 4° C.

### Particle Size Measurement

Particle size analysis was carried out using a Zeta Potential and Particle Size Analyzer (ZetaPals, Brookhaven Instruments Corporation, NY, USA).

### Identification of Protein Content in MVs by Dot-Blot

Exosomes suspended in 5 μl PBS were spotted onto a nitrocellulose membrane (Bio-Rad, CA, USA) and allowed to air-dry. The membrane was treated with 5% (w/v) skim milk (Sigma-Aldrich, MO, USA) in TBS for 1 h at room temperature followed by incubation with the primary antibody. Horseradish peroxidase conjugated secondary antibody and ECL based chemiluminescence system (GE Lifesciences, PA, USA) was used to detect the samples. Images of the membrane were obtained using a ChemiDoc MP system (Bio-Rad, CA, USA).

### RNA, Micro RNA Isolation and Real-Time PCR

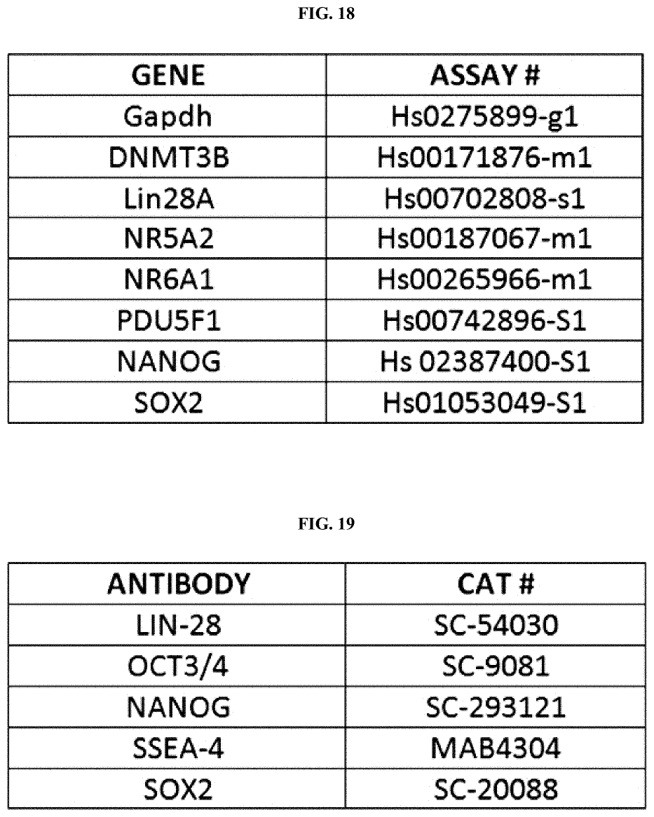

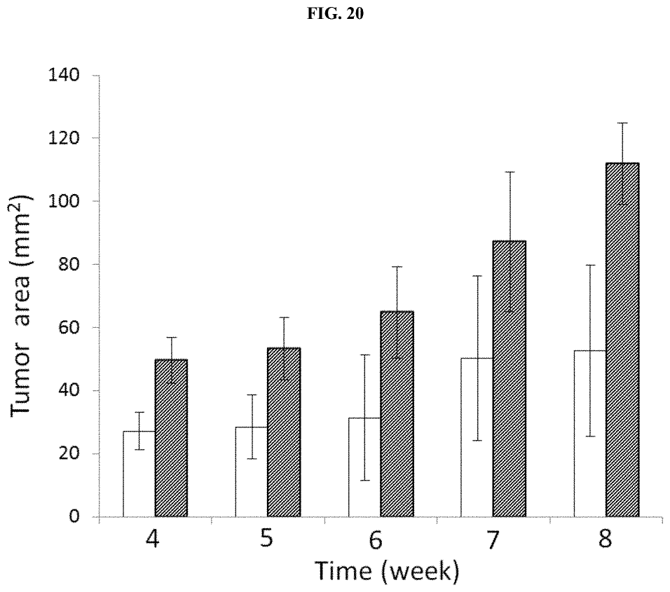

Total RNA from the samples were extracted using RNeasy Mini Kit (Qiagen, CA, USA). The RNA concentration was determined using NanoDrop 2000 (Thermo Scientific, MA, USA). Micro RNA was isolated from the samples using miRNeasy Mini Kit (Qiagen, CA, USA). Real time PCR was performed using Taqman assays (Life Technologies, NY, USA). A list of assays used in the study is provided in FIG. 18.

### Uptake Studies of Exosomes by Human Fibroblasts

The isolated exosomes were labeled with membrane labeling dye PKH67. The labeled exosomes were collected via centrifugation at 100000 g for 70 min, then added to the human fibroblasts with complete growth media. The cellular uptake of exosomes was visualized at specified time intervals by fluorescence microscopy.

Reprogramming of Fibroblasts with Exosomes

IMR-90 cells or primary fibroblasts were cultured on 12-well plates (5×10⁻⁴ cells/well) in respective growth media. DMEM/F12 was supplemented with P/S and 10% FBS. The cells were treated with 300 μl 2i medium of ES-exosome/well for 7-10 days. At indicated time points, cells were washed twice with PBS and collected for analysis.

### Karyotyping of the Reprogrammed Fibroblasts

The reprogrammed cells were cultured for at least 5 passages on Matrigel prior to karyotyping. For karyotyping, the cells were grown on cover glasses coated with Matrigel. Karyotyping was carried out by Parkway Laboratory Services, Singapore.

### Immunohistochemistry

The cells were fixed in formaldehyde for 10 min. After 3 rinses with PBS, the cells were permeablized with PBS containing 0.1% Triton X-100. The non-specific sites were blocked with PBS containing 5% of bovine serum albumin (BSA) for 30 min. Samples were incubated with primary antibody for 2 h at room temperature. Upon 3 washes with PBS containing 1% BSA, the samples were incubated for 45 min with the appropriate antibodies. DAPI was used to stain the nuclei of the cells. Images were taken using IX71 Olympus microscope. The antibodies used for the immunohistochemistry are listed in FIG. 19.

### Embryoid Body and Teratoma Assays

Embryoid body assay was performed to establish the pluripotency of the exosome-induced cells in vitro. Briefly, single cell suspension (1×10⁶/ml) was cultured on ultra-low attachment surfaces for 15 days with DMEM contacting 10% FBS. Total RNA was isolated and screened for differentiation marker genes of the three germ layers by RT-PCR.

Teratoma assays were performed to examine the differentiation potential of the reprogrammed cells in vivo. Briefly, 10 million cells were injected subcutaneously into the NOD/SCID mice. The cells were allowed to grow in the animals for 8 weeks. Explants were harvested and fixed with formalin. The samples were sectioned and analyzed with H&E (Haemotoxylin and Eosin) stain at Histopathology Unit (Biopolis Shared Facilities, Singapore). Experiments involving animals were approved by the IACUC.

### Direct Differentiation to Neural Progenitor Cells

The reprogrammed cells were allowed to undergo direct differentiation to neural lineage progenitors using STEMdiff™ Neural Progenitor Medium, as described by the manufacturer.

Reprogramming of Cancer Cells with Exosomes

Cancer cell lines used in the exosome reprogramming experiments were the breast cancer lines, MCF7 and MDA-MB-231, the colon carcinoma line, DLD-1, and the epidermoid carcinoma line, A431. Each of these cell lines were cultured in the appropriate media, according to suppliers' instructions. 105/mL cells were treated with 300 μl medium of exosomes per well, in 12-well tissue culture plates for 3 days. At indicated time points, cells were washed twice with PBS and collected for analysis. Exosomes were isolated from 4 pluripotent stem cell sources—the embryonic stem cell lines, H1, H7 and H9, and iPS cells derived from IMR-90 fibroblasts.

### Tumorigenicity Assay

In vivo analysis of the tumorigenicity of the reprogrammed cells (using exosomes from H7 embryonic stem cell line) were performed by injecting a 100 μl suspension of 10⁸ MCF7 cells (reprogrammed using exosomes from H7 embryonic stem cell line) into the flank of nude mice. The tumor formation was monitored every week from 4 to 8 weeks. Tumor size was estimated by measuring the long and short axes of the tumors using Vernier caliper. The tumor growth is compared against nude mice injected with control cells, which are MCF7 cells which were not fed with exosomes.

### Drug Resistance Assays

H7-exosome treated and non-treated A431 and MCF7 cancer cells were seeded at a density of 3×10³ cells per well in a 96-well plate, in their respective medium. The anticancer drugs, cisplatin, doxorubicin and sunitinib were then added in concentration series ranging from 0.1 to 1000, 0.001 to 10, and 0.1 to 1000 μg/mL, respectively. After time periods of 24, 48 and 72 hours, the cells were subjected to an Alamar Blue assay for cell viability.

### Scanning Electron Microscopy (SEM) and Transmission Electron Microscopy (TEM)

Scanning Electron Microscopy (SEM) and Transmission Electron Microscopy (TEM) were performed by methods modified from those reported in literature (16).

### Example 2: Reprogramming of Fibroblasts to Exosome-Induced Pluripotent Stem Cells (Ex-iPSCs)

Exosomes are known to contain biological macromolecules. While exosomes isolated from human embryonic stem cells (hESCs) were reported to contain pluripotent stem cell transcription factors (14,17), it is not known whether these exosomes would induce reprogramming.

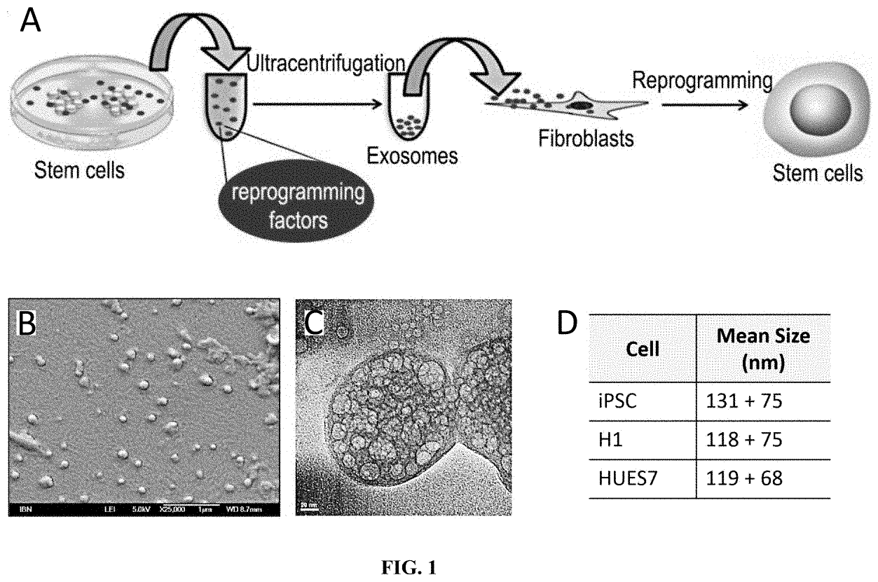

FIG. 1A illustrates the scheme of exosome isolation and reprogramming to obtain iPSC-like cells. Scanning electron microscopy (SEM) (FIG. 1B) and transmission electron microscopy (TEM) (FIG. 10) were performed to investigate the structural characteristics of the exosomes. Under SEM, the exosomes appear to be globular in shape with diameters of in the range of 120-160 nm. Under TEM, the exosomes appear to be composed of multi-vesicular bodies, with a diameter of approximately 140 nm. TEM further shows a clear cell membrane that surrounds the multi-vesicular body.

The exosomes were extracted from an iPSC line (IMR-90) and two hESC lines H1 and HUES7 (hESC) and the sizes were measured by dynamic light scattering. In general the exosomes isolated from pluripotent stem cells were measured to be 102+1.3 nm in size, with a polydispersity index (PDI) of 0.388±0.02 (FIG. 1D). Therefore, the exosomes isolated are intact and show typical structural characteristics of exosomes.

Several RNA species have been reported to induce the reprogramming of somatic cells (18). The presence of these RNAs was examined in exosomes. PCR coupled with reverse transcription was used to identify the presence of messenger RNA (mRNA) in the exosomes. Exosomes were isolated from iPSC (IMR-90), H1 and HUES7 and the relative expression was quantitated by real time PCR (FIG. 2A). Experimental conditions among the exosome group (iPSC-EX, H1-EX and HUES7-EX) were kept identical.

Among the exosome group, exosomes isolated from HUES7 (HUES7-EX) showed higher expression levels of the reprogramming RNAs. To give an insight into the actual amount of these molecules present in the exosomes, the expression level of specified genes in the exosomes were compared to that for total RNA isolated from iPSC (IMR-90), in terms of Ct values (FIG. 2B). Several protein molecules associated with reprogramming were also expressed, which were further examined by dot-blot analysis (FIG. 2C).

Based on the results of the real time PCR and dot blot analysis, it was found that exosomes isolated from different pluripotent stem cell sources exhibited differences in the expression levels of reprogramming proteins in spite of similar culture conditions. Exosomes isolated from HUES-7 cells showed the highest levels of expression of reprogramming proteins. Thus, exosomes isolated from HUES7 were selected to be used for all the subsequent experiments described herein.

The profile of miRNA present in the exosomes from HUES7 was also examined. miRNAs play a critical role in gene regulation and reprogramming. Earlier reports have identified a set of miRNAs that are involved in reprogramming (19). The presence of specific miRNAs in the exosomes by real-time PCR were evaluated, whereupon Let7a, mir125b, mir 182, mir 302b, mir 302d and mir 367 were detected as shown in FIG. 2D.

In summary, pluripotent stem cell-derived exosomes contain proteins, RNAs and miRNAs that are known to induce reprogramming in somatic cells (FIG. 2). Thus, uptake of the exosomes loaded with the abovementioned reprogramming macromolecules could potentially induce the reprogramming of human fibroblasts.

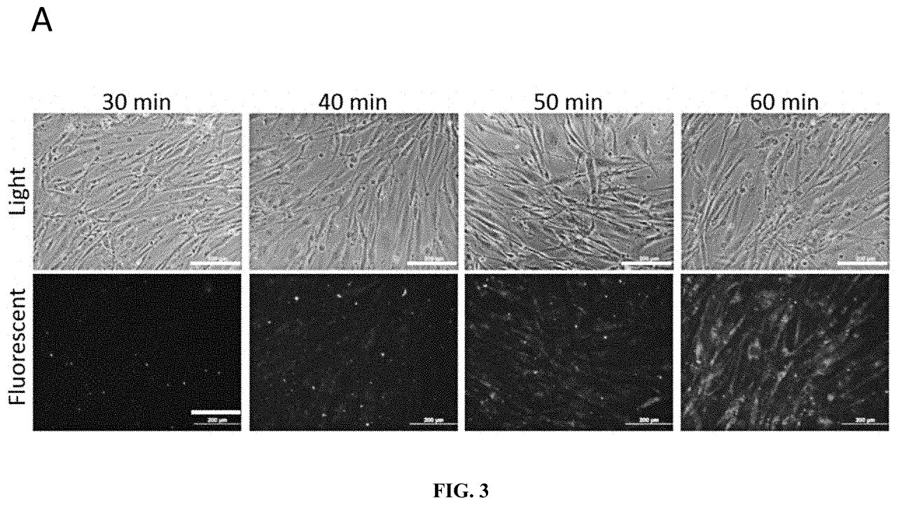

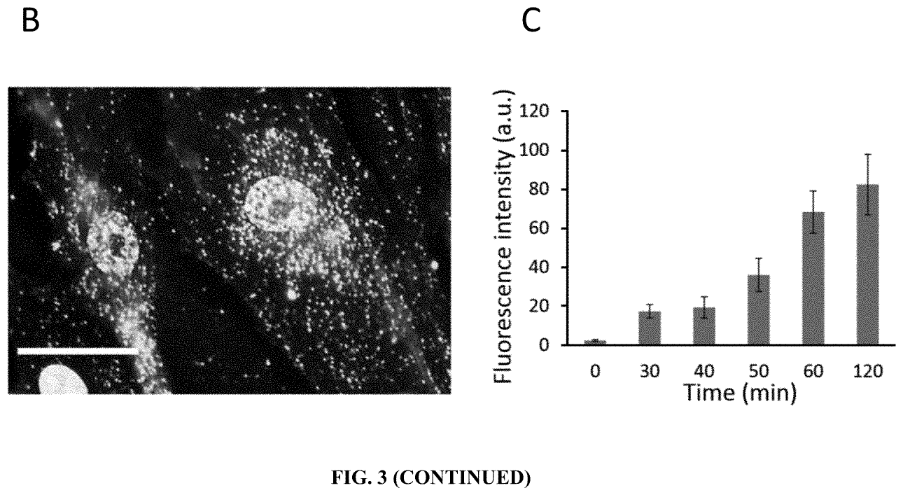

In order to test the uptake of the exosomes, the isolated exosomes from HUES7 were fluorescently labelled using PKH26 cell membrane labeling and the kinetics of the uptake of these exosomes into human fibroblasts were monitored (FIG. 3A). The fluorescently labeled exosomes were indeed taken up by the fibroblasts. Higher magnification fluorescence images show the presence of labeled exosomes inside the cells and more particularly, near the perinuclear region (FIG. 3B). Quantitative fluorescence intensity measurement indicated that the intensity reached a plateau after 120 min of incubation (FIG. 3C). Therefore, the localization analysis confirms the exosomes uptake by human fibroblast was achieved.

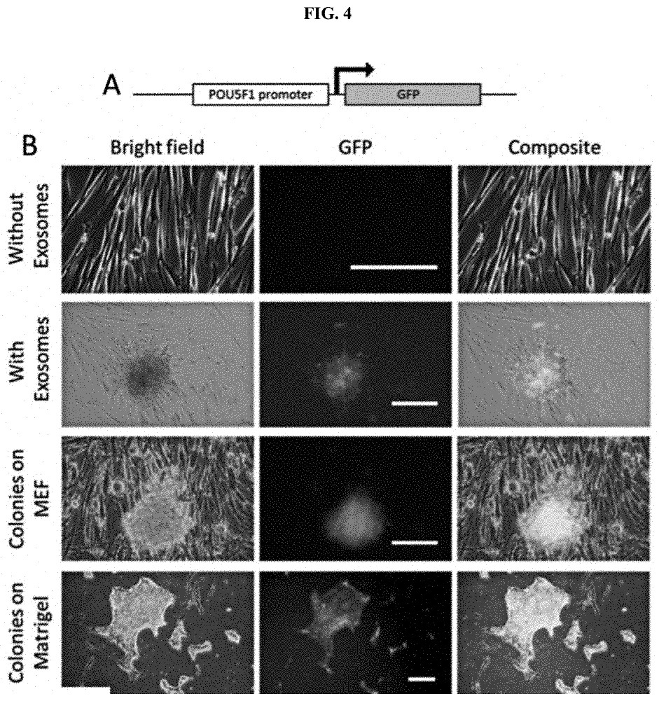

To monitor the reprogramming process, IMR-90 fibroblasts were transfected with POU5f1 (OCT3) promoter-driven GFP vector (FIG. 4A). Exosomes isolated from HUES-7 cells were fed to IMR-90-OCT3-GFP cells. Control cells without exosomes did not show any fluorescence of GFP. By day 3 of exosome treatment, aggregation of cells was observed, and by 10 days of treatment, colonies appeared (FIG. 4B). Several colonies were picked and transferred onto a layer of mitomycin treated mouse embryonic fibroblasts (MEF layer) for further culture. Many of the colonies remained as colonies and never proliferated. However, two colonies that were able to proliferate on MEF layer were identified. These two colonies were subsequently subcultured on Matrigel, where they were continuously expanded. Cell lines derived from these two colonies were designated as EX-iPSC-1 and EX-iPSC-2. Total RNA was collected from these two cell lines grown at passage 0 (P0) and passage 5 (P5) for gene expression analysis. The expression levels of 5 key pluripotent genes at different passages of exosome reprogramming were examined and compared with hESCs and iPSCs. EX-iPSC-1 showed sustained expression of the pluripotent genes at P0 and P5 (FIG. 5). On the other hand, while EX-iPSC-2 showed a similar expression pattern and level of pluripotent genes at P0, the expression of the marker genes were substantially higher at P5 (FIG. 5).

The expression of pluripotent markers at the protein level was ascertained by flow cytometry and immunostaining. Both exosome-induced cell lines, EX-iPSC-1 and EX-iPSC-2 exhibited high levels of Nanog, Oct4 and SOX2, which were expressed at levels comparable to that of the HUES7 cell line (FIG. 6).

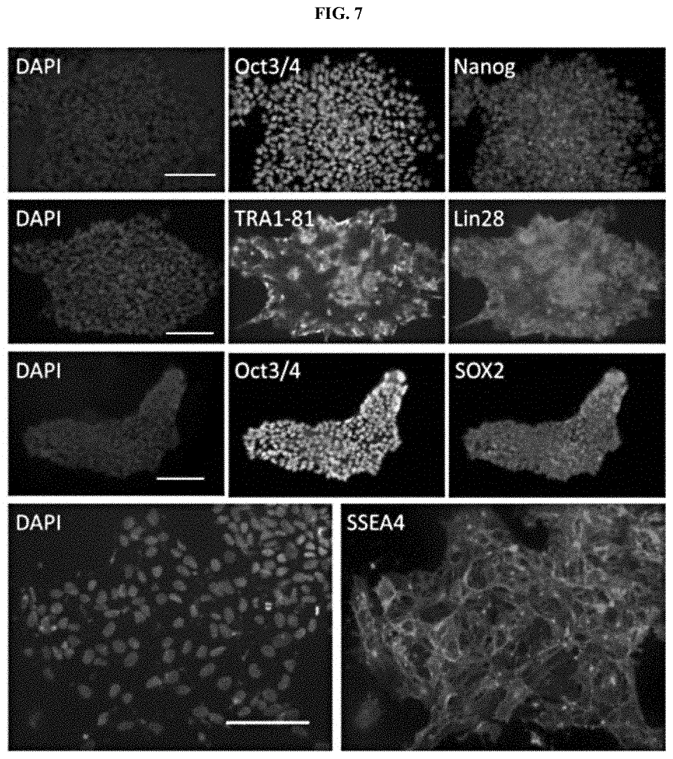

Immunostaining was performed to demonstrate the pluripotent status of the reprogrammed cells, shown for the case of EX-iPSC-2 (FIG. 7). Antibodies against OCT3/4, Nanog, TRA-1-81, Lin28, SOX2 and SSEA-4 were used for immunostaining. Double immunostaining was also performed in some cases to show the co-expression of the pluripotent markers. As expected, nuclear localization was observed for the transcription factors (OCT3/4, Nanog and SOX2). The expression of Lin28 and TRA-1-81 was non-nuclear and localized in the cytoplasm and membrane compartments. On the other hand, immunostaining with SSEA-4 showed the membrane localization of the protein. Similar results were observed with immunostaining of EX-iPSC-1 cells (FIG. 8). Therefore, the immunostaining confirms the pluripotent markers are correctly localized to the respective compartments in the exosomes-reprogrammed cells.

Pluripotent stem cells possess the inherent capacity for differentiation into a variety of cell types. An in vivo teratoma formation assay to verify the pluripotential characteristic of the exosome-reprogrammed cells, which were implanted subcutaneously in mice, was performed. Teratoma formation was observed after 6 weeks of implantation. H&E staining of the sections of the explants were observed under the microscope, and multiple cell types from different lineages were identifiable, such as chondrocytes, lung epithelial cells with blood vessels, intestinal epithelial cells with blood vessels and glandular columnar cells (FIG. 9, FIG. 10).

Directed differentiation of pluripotent stem cells provides therapeutically useful cells for clinical approaches as described above. The ability of the exosome-reprogrammed cells to undergo directed differentiation towards the neural lineage was demonstrated. Success of differentiation was confirmed by positive immunostaining of the cells towards PAX6, nestin, neurofilament and β3 tubulin, as well as upregulated expression of the neural stem markers CD133, MUSASHI, NESTIN, PAX6 and SOX1, as measured by RT-PCR (FIG. 11).

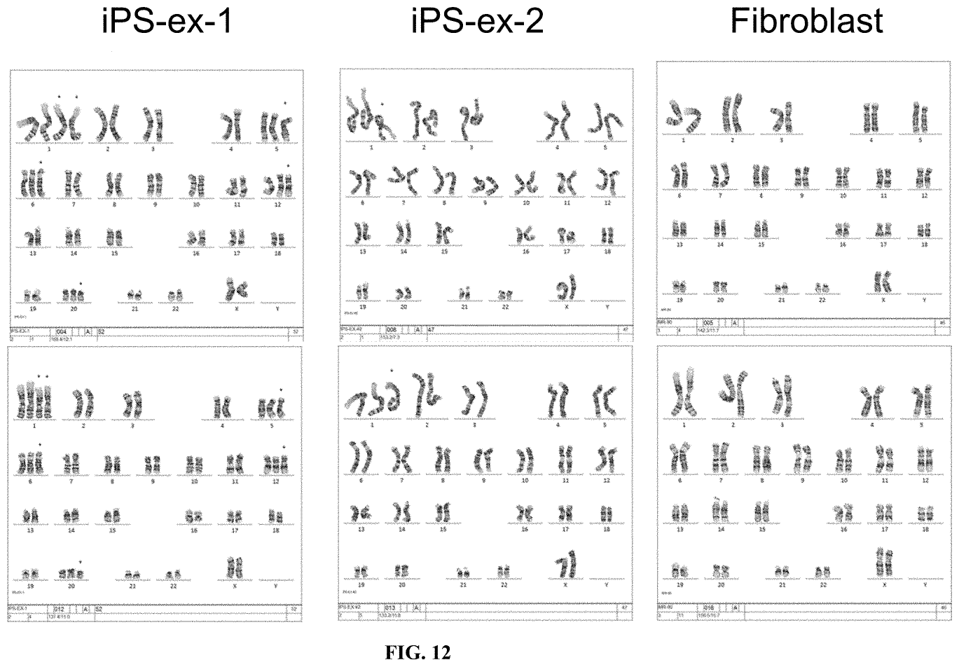

Karyotypic analysis was performed for both the exosome-reprogrammed cells and the fibroblasts from which they were derived (FIG. 12). Chromosomal aberrations were found for both iPS-ex1 and iPS-ex2, while the fibroblast karyotype was normal. iPS-ex1 was associated with 5 aberrations, which were all duplications, whereas iPS-ex2 was associated with 1 aberration, also a duplication. It has been reported that 12-13% of iPSC and hESC cultures have abnormal karyotypes (20), while aneuploidy was found to be responsible for 20%-53% of arrested of iPSC colonies (21). Despite being aneuploid, the two exosome-reprogrammed cell lines derived in the present study were able to proliferate and undergo differentiation in a manner similar to typical pluripotent stem cell cultures.

### Example 3: Reprogramming of Cancer Cells to EX-iCSCs

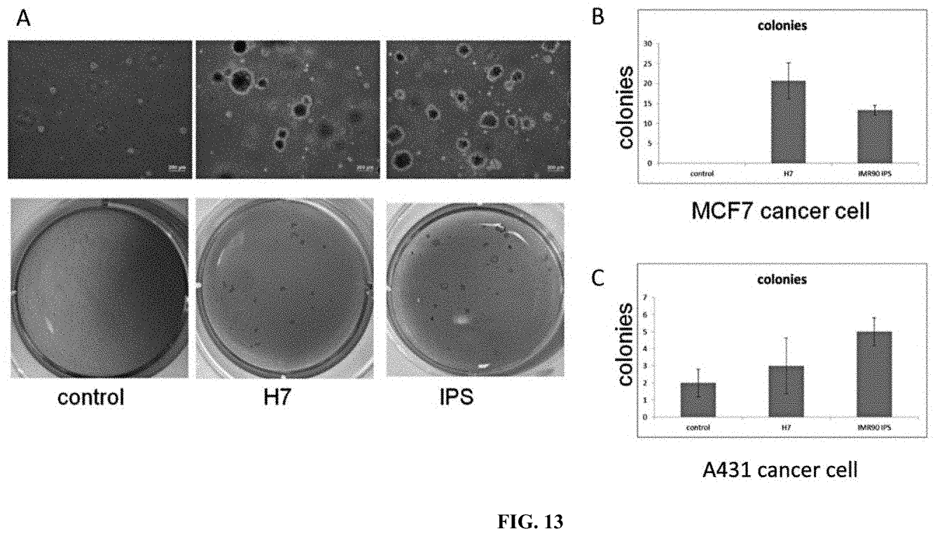

A colony formation assay on soft agar was performed to evaluate self-renewal ability of the EX-iCSCs (FIG. 13A). Treatment with exosomes from iPS cells significantly increased the number of colony-like cell clusters for both the MCF7 (FIG. 13B) and A431 cancer cell lines (FIG. 13C), while treatment with ES cell-derived exosomes increased the clonogenic ability of the MCF7, but not A431 cancer cell line.

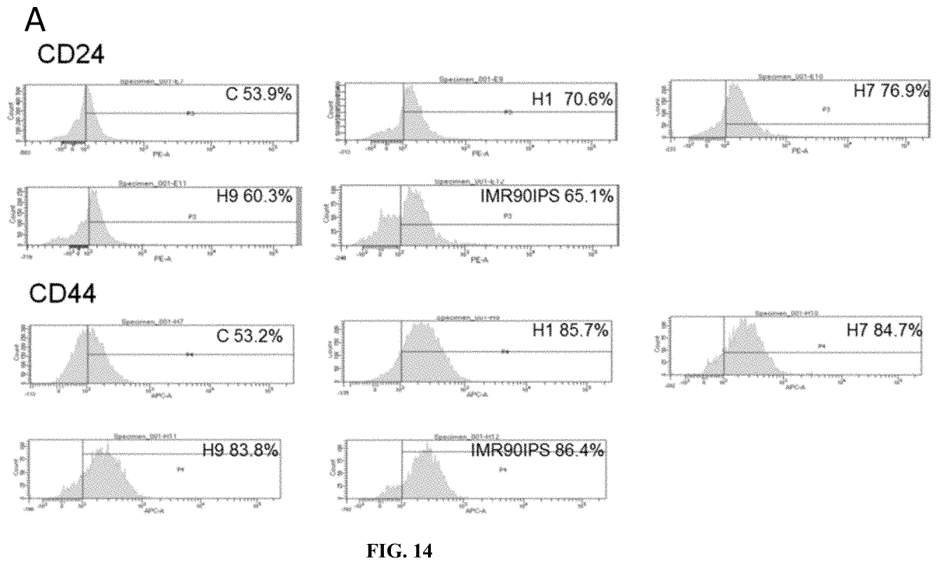

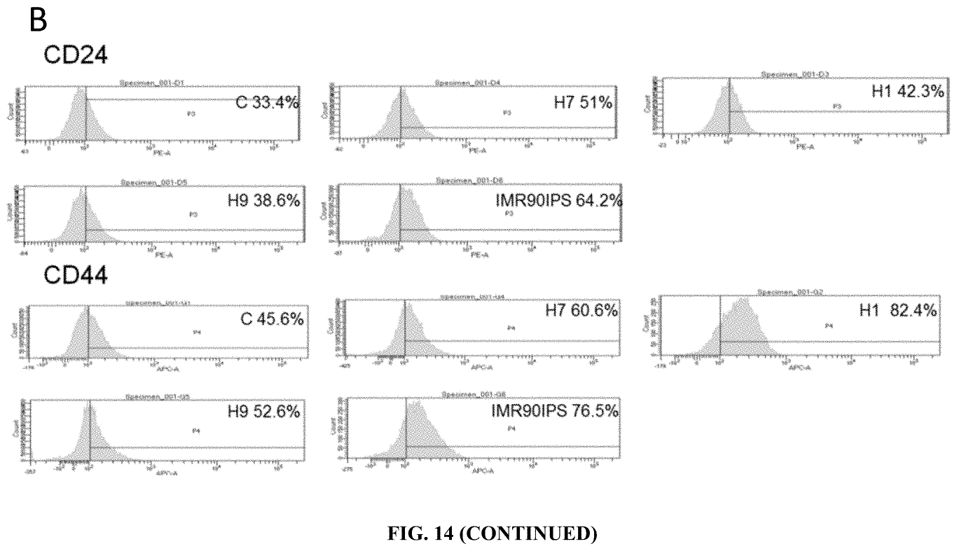

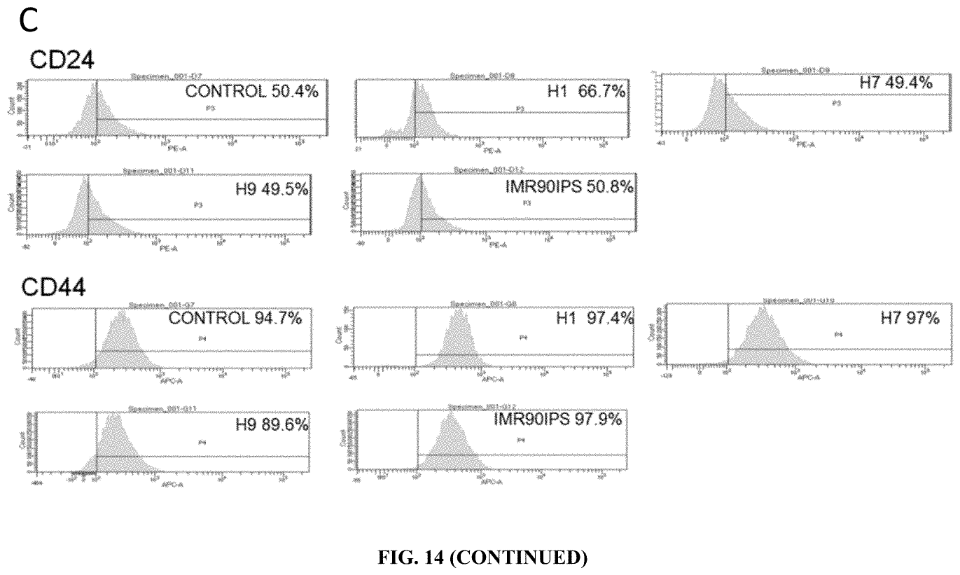

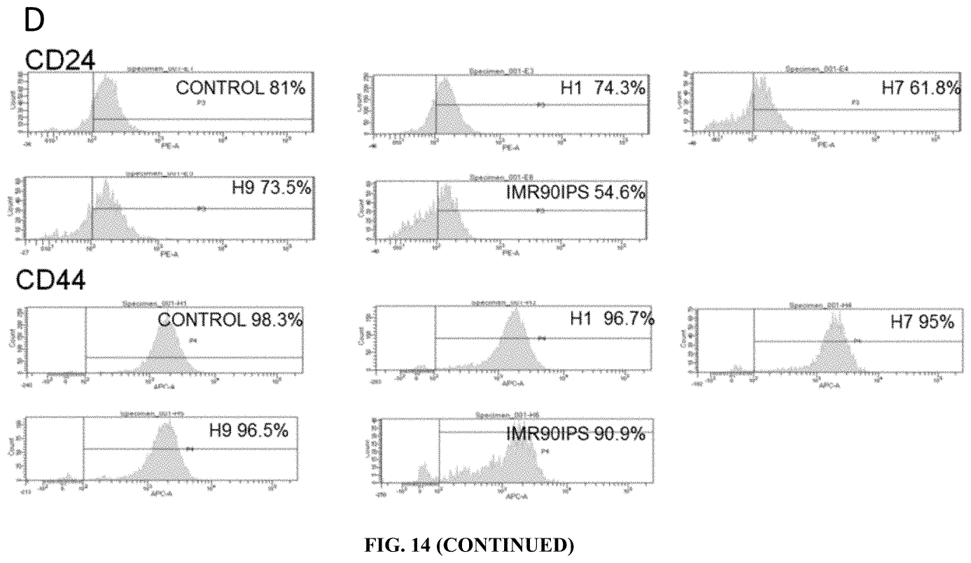

To test whether the EX-iCSCs were enriched in CSC-like properties, the activity of CD24 and CD44, which are specific cancer stem cell (CSC)-related surface markers, were determined by flow cytometry (FIG. 14). The results are summarized in FIG. 15. Flow cytometry analysis showed higher CD44 and CD24 activity of the exosome-treated cancer cells, to different degrees. For the MCF7 cell line, higher CD44 and CD24 activity was observed for cells subjected to treatment with all exosome groups. The exosome treated A431 cells contained significantly higher CD24 and CD44 positive cell populations, except for the case of H9-derived exosomes. Finally, for the DLD-1 cell line, a higher proportion of CD24 positive cells was observed only for the H1 exosome-treated group. For the MDA-MB-231 cancer cell line, no significant changes were observed in the CD24 or CD44 populations were observed upon exosome treatment.

Drug resistance of EX-iCSCs

Drug resistance has been associated with stem cell-like traits in cancer cells. The MCF7 and A431 cell lines were treated with exosomes to induce EX-iCSCs, following which they were subjected to viability (Alamar Blue) assays (FIG. 16, FIG. 17). Exosome-treated cells exhibited significantly higher viability at all time points especially for A431 EX-iCSCs treated with Sunitinib, and MCF7 EX-iCSCs treated with doxorubicin. These results show that the exosome-treated cells may have acquired the stem cell-associated characteristic of drug resistance. Thus, as the exosome-treated cells may be more resistant to sunitinib and doxorubicin compared to non-treated cells, drugs identified through screening procedures that selectively kill the exosome-induced stem cell-like cancer cells would likely be more effective in killing the cancer stem cell/tumor-initiating cancer cell population in vivo.

### REFERENCES

1. Sabin K, Kikyo N. Microvesicles as mediators of tissue regeneration. Transl Res. 163(4) (2014), 286-95.
2. Bian S, Zhang L, Duan L, Wang X, Min Y, Yu H. Extracellular vesicles derived from human bone marrow mesenchymal stem cells promote angiogenesis in a rat myocardial infarction model. J Mol Med. 92 (2014), 387-397.
3. Nakano M, Nagaishi K, Konari N, Saito Y, Chikenji T, Mizue Y, Fujimiya M. Bone marrow-derived mesenchymal stem cells improve diabetes-induced cognitive impairment by exosome transfer into damaged neurons and astrocytes. Scientific Reports 6 (2016), 24805.
4. Ma S, Xie N, Li W, Yuan B, Shi Y, Wang Y. Immunobiology of mesenchymal stem cells. Cell Death Differ. 21(2) (2014), 216-25.
5. Prockop D J, Youn Oh J. Mesenchymal Stem/Stromal Cells (MSCs): Role as Guardians of Inflammation. Mol Ther. 20(1) (2012), 14-20.
6. Takahashi K, Yamanaka S. Induction of pluripotent stem cells from mouse embryonic and adult fibroblast cultures by defined factors. Cell 126(4) (2006), 663-76.
7. Chang C-W, Lai Y-S, Pawlik K M, Liu K, Sun C-W, Li C, Schoeb T R, Townes T M. Polycistronic Lentiviral Vector for “Hit and Run” Reprogramming of Adult Skin Fibroblasts to Induced Pluripotent Stem Cells. Stem Cells 27(5) (2009), 1042-9.
8. Stadtfeld M, Nagaya M, Utikal J, Weir G, Hochedlinger K. Induced pluripotent stem cells generated without viral integration. Science 322(5903) (2008), 945-9.
9. Khan M, Narayanan K, Lu H, Choo Y, Du C, Wiradharma N, Yang Y-Y, Wan A C. Delivery of reprogramming factors into fibroblasts for generation of non-genetic induced pluripotent stem cells using a cationic bolaamphiphile as a non-viral vector. Biomaterials 34(21) (203), 5336-43.
10. Kim D, Kim C H, Moon J I, Chung Y G, Chang M Y, Han B S, Ko S, Yang E, Cha K Y, Lanza R, Kim K S. Generation of human induced pluripotent stem cells by direct delivery of reprogramming proteins. Cell Stem Cell 4(6) (2009), 472-6.
11. Zhou H, Wu S, Joo J Y, Zhu S, Han D W, Lin T, Trauger S, Bien G, Yao S, Zhu Y, Siuzdak G, Scholer H R, Duan L, Ding S. Generation of induced pluripotent stem cells using recombinant proteins. Cell Stem Cell 4(5) (2009), 381-4.
12. Miyoshi N, Ishii H, Nagano H, Haraguchi N, Dewi D L, Kano Y, Nishikawa S, Tanemura M, Mimori K, Tanaka F, Saito T, Nishimura J, Takemasa I, Mizushima T, Ikeda M, Yamamoto H, Sekimoto M, Doki Y, Mori M. Reprogramming of mouse and human cells to pluripotency using mature microRNAs. Cell Stem Cell 8(6) (2011), 633-8.
13. Warren L, Manos P D, Ahfeldt T, Loh Y H, Li H, Lau F, Ebina W, Mandal P K, Smith Z D, Meissner A, Daley G Q, Brack A S, Collins J J, Cowan C, Schlaeger T M, Rossi D J. Highly efficient reprogramming to pluripotency and directed differentiation of human cells with synthetic modified mRNA. Cell Stem Cell 7(5) (2010), 618-30.
14. Zhou J, Ghoroghi S, Benito-Martin A, Wu H, Unachukwu U J, Einbond L S, Guariglia S, Peinado H, Redenti S. Characterization of Induced Pluripotent Stem Cell Microvesicle Genesis, Morphology and Pluripotent Content. Scientific Reports 6 (2016), 19743.
15. Goldberg L, Aliotta J, Chatterjee D, Stewart C, Carpanetto A, Collino F, Bruno S, Camussi G, Quesenberry P. Mesenchymal stromal cell-derived extracellular vesicles rescue radiation damage to murine marrow hematopoietic cells. Wen S, Dooner M, Cheng Y, Papa E, Del Tatto M, Pereira M, Deng Y. Leukemia 30(11) (2016), 2221-2231.
16. Wu Y, Deng W, Klinke D J 2nd. Exosomes: improved methods to characterize their morphology, RNA content, and surface protein biomarkers. Analyst 140(19) (2015), 6631-42.
17. Shufeng Zhou, Mohamed Abdouh, Vincenzo Arena, Manuel Arena, Goffredo Orazio Arena. Reprogramming Malignant Cancer Cells toward a Benign Phenotype following Exposure to Human Embryonic Stem Cell Microenvironment. PLoS One 12(1) (2017), e0169899.
18. Warren L, Manos P D, Ahfeldt T, Loh Y H, Li H, Lau F, Ebina W, Mandal P K, Smith Z D, Meissner A, Daley G Q, Brack A S, Collins J J, Cowan C, Schlaeger T M, Rossi D J. Highly efficient reprogramming to pluripotency and directed differentiation of human cells with synthetic modified mRNA. Cell Stem Cell 7(2010), 618-30.
19. Frederick Anokye-Danso, Melinda Snitow, Edward E. Morrisey. How microRNAs facilitate reprogramming to pluripotency. J Cell Sci. 125(18) (2012), 4179-4787.
20. Taapken S M, Nisler B S, Newton Mass., Sampsell-Barron T L, Leonhard K A, McIntire E M, Montgomery K D. Karotypic abnormalities in human induced pluripotent stem cells and embryonic stem cells. Nat Biotechnol. 29(4) 2011, 313-4.
21. Yu Y, Chang L, Zhao H, Li R, Fan Y, Qiao J. Chromosome microduplication in somatic cells decreases the genetic stability of human reprogrammed somatic cells and results in pluripotent stem cells. Sci Rep. 5 (2015) 10114.

1. A method of reprogramming a differentiated cell into an undifferentiated cell state, comprising introducing an exosome from an undifferentiated cell into the differentiated cell.

2. The method of claim 1, wherein the undifferentiated cell is one or more of the following:
(a) a stem cell;
(b) a stem cell selected from the group consisting of embryonic stem cell (ESC), induced pluripotent stem cell (iPSC), and embryonic germ cell;
(c) a cell from a mammal;
(d) a cell from a human;
(e) an embryonic stem cell obtained from a cell line selected from the group consisting of H1, H7, H9, and HUES-7 cell lines; and,
(f) an induced pluripotent stem cell which is IMR-90-iPSC.

3.-7. (canceled)

8. The method of claim 1, wherein the differentiated cell is a somatic cell or a differentiated cancer cell.

9. The method of claim 8, wherein the somatic cell is one or more of the following:
(a) a somatic cell selected from the group consisting of fibroblast, somatic stem cell, sertoli cell, endothelial cell, neuron, pancreatic islet cell, epithelial cell, hepatocyte, hair follicle cell, keratinocyte, hematopoietic cell, melanocyte, chondrocyte, lymphocyte, erythrocyte, macrophage, monocyte, mononuclear cell, muscle cell, and combinations thereof;
(b) a fibroblast selected from the group consisting of lung fibroblast, dermal fibroblast, bladder fibroblast, uterine fibroblast, vas deferens fibroblast, and combinations thereof;
(c) a lung fibroblast which is human lung fibroblast IMR-90; and
(d) an epithelial cell which is granulosa epithelial, or a muscle cell which is cardiac muscle cell.

10.-12. (canceled)

13. The method of claim 8, wherein the cancer cell is one or more of the following:
(a) a breast cancer cell, colorectal cancer cell, epidermoid cancer cell, epithelial tissue cancer cell (carcinoma cell), connective tissue cancer cell (sarcoma cell), blood cancer cell, cancer cell from the lymphatic system (lymphoma cell), lung cancer cell, skin cancer cell and combinations thereof;
(b) a breast cancer cell selected from the group consisting of MCF7, MDA-MB-231, and BT474 cell; a colorectal cancer cell selected from the group consisting of colon carcinoma cell, rectal cancer cell and colorectal sarcoma cell; an epidermoid cancer cell selected from the group consisting of A431, and AW8507 cell; a carcinoma cell selected from the group consisting of adenocarcinoma cell, basal cell carcinoma cell, squamous cell carcinoma cell, and transitional cell carcinoma cell; a sarcoma cell selected from the group consisting of soft tissue sarcoma cell, chondrosarcoma cell, rhabdomyosarcoma cell, and leiomyosarcoma cell; a blood cancer cell selected from the group consisting of acute lymphocytic leukemia cell, acute myelogenous leukemia cell, chronic lymphocytic leukemia cell, and chronic myelogenous leukemia cells; a lymphoma cell selected from the group consisting of Hodgkin lymphoma cell and non-Hodgkin lymphoma cell, and a skin cancer cell selected from the group consisting of superficial spreading melanoma cell, lentigo maligna cell, acral lentiginous melanoma cell and nodular melanoma cell; and
(c) a colon carcinoma cell selected from the group consisting of DLD-1, SW1116, Caco-2, SW480, and combinations thereof.

14.-15. (canceled)

16. The method of claim 1, wherein the exosome contains one or more of the following:
(a) or more pluripotent factors selected from the group consisting of NANOG, OCT3/4, SOX2, FGF2, NR5a2, SSEA-4, TR-1-60, TR-1-81, and combinations thereof; and
(b) one or more miRNAs selected from the group consisting of Let7a, mir-125b, mir-145, mir-182, mir-302b, mir-302d, mir-367, and combinations thereof.

17. (canceled)

18. The method of claim 1, wherein the undifferentiated cell state is a pluripotent cell state or a cancer stem cell-like cell state.

19. The method of claim 18, wherein the cell in a pluripotent cell state has one or more of the following characteristics:
(i) has a similar gene and surface marker expression profile as that of an embryonic stem cell and an induced pluripotent stem cell;
(ii) is pluripotent; and
(iii) is able to form three germ layer tissues.

20. The method of claim 18, wherein the cell in a cancer stem cell-like cell state has one or more of the following characteristics:
(i) has a similar gene and surface marker expression profile as a cancer stem cell; and
(ii) has similar drug resistance characteristics as a cancer stem cell.

21. The method of claim 1, wherein the step of introducing the exosome into the differentiated cell comprises contacting the differentiated cell with the exosome from the undifferentiated cell in a first medium suitable for the uptake of the exosome by the differentiated cell, to thereby reprogram the differentiated cell into the undifferentiated cell state.

22. The method of claim 21, wherein the first medium comprises Dulbecco's Modified Eagle Medium/Nutrient Mixture F-12 (DMEM/F12), Penicillin-Streptomycin (P/S) and 10% Fetal Bovine Serum (FBS).

23. The method of claim 21, wherein the contacting in the first medium is for at least 4 hours or more, at least 3 days or more, at least 10 days or more, or at least 14 days or more.

24. The method of claim 21, comprising incubating the reprogrammed cell in a second medium, wherein the second medium is selected from the group consisting of ESGRO-2i and DMEM.

25. The method of claim 24, wherein the incubation in the second medium is for at least 3 hours to at least 14 days.

26. (canceled)

27. A method of screening for a candidate agent suitable for cancer treatment, comprising contacting an undifferentiated cell population obtained using the method of claim 1 with a candidate agent and determining the ability of the candidate agent to inhibit the growth of the undifferentiated cell population or kill the undifferentiated cell population.

28. A method of treating a condition, comprising:
differentiating an undifferentiated cell population obtained using the method of claim 1 into a desired specific differentiated cell population; and
transplanting the differentiated cell population to a subject in need thereof to address a functional deficit in damaged or diseased tissues of the same type or characteristics.

29. The method of claim 28, wherein the condition is one or more of the following: (a) spinal cord defect and (b) Parkinson's disease.
